(19) United States
(12) Patent Application Publication
(10) Pub. No.: US 2010/0209377 A1
(43) Pub. Date: Aug. 19, 2010

(54) HAIR STYLING AND CONDITIONING PERSONAL CARE FILMS

(76) Inventors: Tatiana V. Drovetskaya, Basking Ridge, NJ (US); Wei Hong Yu, Edison, NJ (US); Edward F. DiAntonio, Staten Island, NY (US); Susan L. Jordan, Doylestown, PA (US)

Correspondence Address: UNION CARBIDE CHEMICALS AND;PLASTICS TECHNOLOGY CORPORATION, P.O. Box 1967, Midland, MI, 48641-1967, US

(21) Appl. No.: 12/665,254

(22) PCT Filed: Jun. 26, 2008
(86) PCT No.: PCT/US08/68366

§ 371 Date: Apr. 13, 2010

Related U.S. Application Data
(60) Provisional application No. 60/947,141, filed on Jun. 29, 2007.

(51) Int. Cl. A61K 8/73; A61Q 5/06

(52) U.S. Cl. 424/70.13

(57) ABSTRACT

The present invention provides personal care compositions in film form having less than about 30 weight percent water-soluble chitosan derivative and greater than about 40 weight percent of a water soluble film forming agent.

### FIELD

The present invention relates to personal care films for use in hair styling or conditioning.

### BACKGROUND

Personal care films are an exciting development in the hair care industry. In theory, such films allow the styling power of an array of cans and bottles to be conveyed to a use site in a pocket-size package. At the use site, the film can be wetted with an aqueous liquid to form a wet product that can then be applied to the hair. Unfortunately, this unparalleled portability has never been optimized.

Currently available styling films demonstrate poor “in hands” properties. For example, they can be tacky, as a result of a combination of polymers and plasticizers present. Similarly, some currently available styling films do not dissolve fast enough and therefore can feel grainy, lumpy, or stringy as a result of relatively long disintegration times.

The success of a cosmetic, including personal care films, depends in great measure on the way it feels to a user at the time of use. Remedying the current drawbacks to currently available films is not straightforward. For example, merely removing tack-causing ingredients is not an option as it may destroy the film's styling/fixative performance and/or negatively impact its mechanical properties. Similarly, lowering disintegration times can result in a wet product that runs through the fingers instead of being appropriately viscous and may actually increase tackiness.

Another challenge in the styling industry is to create products that do not flake off the hair upon drying. Combing, or in some cases just touching, the hair can result in the appearance of flakes that can look like dandruff and are unsightly. For dissolvable styling films, the concentrated product form results in gels with relatively higher content of film-forming materials which exacerbates flaking.

Furthermore, to optimize in-use properties and dissolution time, formulators of dissolvable films often have to leave out or reduce the amount of the “traditional” conditioning agents, such as, for example, polymers, cationic surfactants, and/or silicones. This approach yields styling products that do not offer in-situ conditioning, exhibited as, for example, improved hair feel, comb, and/or mitigation of electrostatic fly-away. Conditioning is highly desirable to the consumer and greatly contributes to the overall grooming experience.

Thus, what is needed are new types of personal care films with better in hands properties and improved multifunctional performance on hair.

### SUMMARY

In one embodiment, the present invention provides personal care dissolvable films, comprising less than about 30 weight percent water-soluble chitosan derivative and greater than about 40 weight percent of a water soluble film forming agent comprising at least one of cellulose derivatives or pullulans.

### DETAILED DESCRIPTION

In one embodiment, the present invention provides a personal care dissolvable film, comprising less than about 30 weight percent water-soluble chitosan derivative and greater than about 40 weight percent of a water soluble film forming agent comprising at least one of cellulose derivatives or pullulans.

“Weight percent” refers to the weight of the component in a theoretical completely dried film, in other words, as if the film had been dried until only nonvolatile components remained. Thus, for this application, 30 weight percent is independent of humidity.

The water-soluble chitosan derivative includes anionic, cationic, amphoteric or nonionic chitosan polymers. In one embodiment, the water-soluble chitosan derivative is a chitosan salt of pyrrolidone carboxylic acid, which is, for example available under the trade name of KYTAMER PC from The Dow Chemical Company. Chitosan PCA salt is known for its moisturization properties, in fact, it has been described by some as a film plasticizer.

In one embodiment, the water-soluble chitosan derivative is present in the personal care dissolvable film in a range from about 5 weight percent to about 30 weight percent. In one embodiment, the water-soluble chitosan derivative is present in a range from about 10 weight percent to about 25 weight percent. In one embodiment, the water-soluble chitosan derivative is present in a range from about 20 weight percent to about 30 weight percent. In one embodiment, the water-soluble chitosan derivative is present in a range from about 22 weight percent to about 27 weight percent. The recited ranges are given to amply illustrate certain features of the invention; however, additional ranges are understood to be contemplated. Indeed, all novel combinations and subcombinations found within the above ranges are contemplated and may be placed in the appended claims.

In one embodiment, the water soluble film forming agent is a pullulan. In one embodiment, the water soluble film forming agent is a cellulose ether based polymer. In one embodiment, the water soluble film forming agent is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose. In a preferred embodiment, the water soluble film forming agent is hydroxypropyl methylcellulose.

When present, the water soluble film forming agent is present in an amount from about 40 weight percent to about 95 weight percent of the dry film. In one embodiment, the water soluble film forming agent is present in an amount from about 40 weight percent to about 80 weight percent. In one embodiment, the water soluble film forming agent is present in an amount from about 45 weight percent to about 65 weight percent.

In one embodiment, the film further comprises a cosmetically acceptable plasticizer. “Cosmetically acceptable” refers to ingredients typically used in personal care compositions, and is intended to underscore that materials that are toxic, irritating, or unpleasant smelling when present in the amounts typically found in personal care compositions are not contemplated as part of the present invention. Plasticizers include any of the plasticizers listed in McCutcheon's Functional Materials (1992). Preferably, the plasticizer is lipid, a polyol, an acid, a polyester, or water-soluble organopolysiloxane.

Examples of lipid plasticizers include waxes (such as ethoxylated jojoba or beeswax), mineral oils, paraffin derivatives, vegetable oils, triglycerides, lanolins, unsaturated fatty acids, and their derivatives.

Examples of polyol plasticizers include glycerin, ethylene glycol, propylene glycol, sugar alcohols (such as sorbitol, SORBETH-30, manitol, maltitol, lactitol), saccharides (such as fructose, glucose, sucrose, maltose, lactose, and high fructose corn syrup), polysaccharides, ascorbic acid, decyl glucoside, propylene glycol, polyethylene glycol, PEG derivatives (ether, ester), and dimethicone copolyols (such as PEG-12 dimethicone, PEG/PPG-18/18 dimethicone, and PPG-12 dimethicone).

Examples of acid plasticizers include carboxylic acids (such as citric acid, maleic acid, succinic acid, adipic acid, azelaic acid, benzoic acid, dimer acids, fumaric acid, isobutyric acid, isophthalic acid, lauric acid, linoleic acid, maleic acid, maleic anyhydride, melissic acid, myristic acid, oleic acid, palmitic acid, phosphoric acid, phthalic acid, ricinoleic acid, sebacic acid, stearic acid, succinic acid, 1,2-benzenedicarboxylic acid polyacrylic acid, and polymaleic acid), alpha and beta hydroxy acids (such as glycolic acid, lactic acid (including sodium, ammonium, and potassium salts), and salicylic acid), and sulfonic acid derivatives.

Examples of polyester plasticizers include glycerol triacetate, acetylated-monoglyceride, diethyl phthalate, triethylcitratetriethyl citrate, tributyl citrate, acetyl triethyl citrate, acetyl tributyl citrate, acetyl triethylcitrate, diisobutyl adipate, butyl stearate, and phthalates.

In one embodiment, the plasticizer includes a mixture of at least two plasticizers. In one embodiment, the plasticizer includes at least two of small molecule polyol, polyethylene glycol derivative of dimethicone, and alkyl glucoside.

The plasticizer, when present, is present in an amount from about 0.01 to about 30 weight percent, that is, by the weight of the plasticizer in a theoretical completely dried film (as if the film had been dried until only nonvolatile components remained). In a preferred embodiment, the plasticizer is present in an amount from about 5 to about 25 weight percent. In one embodiment, the plasticizer is present in an amount from about 10 to about 20 weight percent. In one embodiment, the plasticizer is present in an amount of about 16 weight percent.

Composition of the present invention can further incorporate other ingredients known in the art of hair care formulations and dissolvable films. Other optional ingredients for personal care compositions of the present invention include at least one of the following: additional film forming agents, cosmetically acceptable emollients, moisturizers, conditioners, oils, sunscreens, surfactants, emulsifiers, preservatives, rheology modifiers, colorants, preservatives, pH adjustors, propellants, reducing agents, fragrances, foaming or de-foaming agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides.

The moisturizers include 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, and polyols, such as, for example, propylene glycol, glycerol and sorbitol.

Conditioners include stearalkonium chloride, dicetyldimonium chloride, lauryl methyl gluceth-10 hydroxypropyldimonium chloride, and natural and synthetic conditioning polymers such as polyquaternium-4, polyquaternium-7, polyquaternium-10, polyquaternium-24, polyquaternium-67 and the like, chitosan and derivatives thereof.

Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylpolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID 350 silicone fluid (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes from the list above may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances may be aldehydes, ketones, or oils obtained by extraction of natural substances or synthetically produced as described above. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

### EXAMPLES

The following examples are for illustrative purposes only and are not intended to limit the scope of the present invention. All percentages are by weight unless otherwise specified.

### Example 1

Exemplary personal care compositions contain the components recited in TABLE 1.

TABLE 1

| # | Component | Batch 1 | Batch 2 |
|---|---|---|---|
| 1 | METHOCEL E3 hydroxypropyl methyl- | 7.0 | 6.3 |
| | cellulose | | |
| | Pullulan | — | 0.7 |
| | Wt. % film forming agent (in final | 56% | 59% |
| | dry film) | | |
| 2 | KYTAMER PC chitosan/pyrrolidone | 60.0 | 60.0 |
| | carboxylic acid salt (5% soln) | | |
| | (Wt. % (in final dry film)) | (24 wt %) | (25 wt %) |
| 3 | Glycerin | 0.50 | 0.50 |
| 4 | DC-193 PEG-12 Dimethicone | 0.50 | 0.50 |
| 5 | Citric Acid (10% soln) | 4.0 | 4.0 |
| 6 | PLANTAREN 2000 Decyl Glucoside (50% | 2.00 | 1.00 |
| | solution) | | |
| 7 | Deionized Water | balance | balance |

All numerals without parentheses are in grams.

The parentheticals are provided to illustrate that the films in TABLE 1 all have less than 30 weight percent chitosan/pyrrolidone carboxylic acid salt and more than 40 weight percent film forming agent in the resulting dry film. Though provided for convenience, the weight percent can also be calculated by dividing the weight of the dry KYTAMER PC component (a) by the total weight of the remaining non-volatile ingredients in the film pre-mix (b) where:
(a)=5% of the total weight of the KYTAMER PC aqueous solution used, and
(b)=Weight (1)+(a)+Weight (3)+Weight (4)+0.1*Weight (5)+0.5*Weight (6)
   The weight percent of film forming materials other than KYTAMER PC aqueous solution can also be calculated by dividing the weight of all these film forming components (c) by the total weight of remaining non-volatile ingredients in the film pre-mix (b) where:
(c)=Weight (1)¹ ¹METHOCEL E3 and pullulan
(b)=(c)+(a)+Weight (3)+Weight (4)+0.1*Weight (5)+0.5*Weight (6)

The ingredients are combined into a liquid pre-mix formulation for each batch. Three drops of GLYDANT preservative were added at the end to each formulation. The liquid pre-mix formulations are then cast by drawing down and drying overnight at room temperature to afford 2 mils thick films.

### Example 2

### Comparative

Comparative compositions contain the components recited in TABLE 2.

TABLE 2

| | Compara- | Compara- | Compara- | Compara- |
|---|---|---|---|---|
| | tive | tive | tive | tive |
| Component | Sample A | Sample B | Sample C | Sample D |
| METHOCEL E3 | — | 2.4 | 10 | — |
| hydroxypropyl | | | | |
| methylcellulose | | | | |
| Pullulan | — | — | — | 10 |
| KYTAMER PC | 97.56 | 72.0 | — | — |
| chitosan/pyrrolidone | | | | |
| carboxylic acid salt | | | | |
| (5% soln) | | | | |
| Glycerin | 0.24 | 0.30 | 0.50 | 0.50 |
| DC-193 PEG-12 | 0.24 | 0.30 | 0.50 | 0.50 |
| Dimethicone | | | | |
| Citric Acid (10% | 0.98 | 2.40 | 4 | 4 |
| soln) | | | | |
| PLANTAREN 2000 | 0.98 | 1.20 | 2 | 2 |
| Decyl Glucoside | | | | |
| (50% solution) | | | | |
| Deionized Water | 0 | 23.8 | 83 | 83 |

Sample A contains 82% of chitosan PCA salt (dry film base) and no cellulosic derivatives or pullulan. Sample B contains 48% of chitosan PCA salt (dry film base) and 32 wt. % of METHOCEL. Samples C and D contain no chitosan PCA salt.

Samples A and B are covered in a concurrently filed US provisional application (Atty Docket 66168), the entire disclosure of which is incorporated herein by reference in its entirety.

All numerals are in grams. The ingredients are combined into a liquid pre-mix formulation for each sample. Three drops of GLYDANT preservative were added at the end to each formulation. The liquid pre-mix formulations are then cast by drawing down and drying overnight at room temperature in a vessel of sufficient area to afford 2 mils thick films.

### Example 3

In-situ formulations were prepared by dissolving 0.3 g of dry film (made substantially according to the protocol of Example 1 and Example 2, and representing Batch 1, Batch 2, Comparative Samples C and D in 12 g of water and stirring until complete dissolution was achieved. Then, 0.4 g of the in situ gel was applied to pre-wetted, pre-combed, eight inch long, ˜4.5 g tresses of European virgin brown hair using a pipette in small portions, evenly from top (swatched end) to bottom (hair tips). The gel was then worked into the hair with fingers going from top to bottom of each tress five times. The tress was then reversed and the same procedure was repeated five more times. At the end, the tress was combed to eliminate knots, smoothed with fingers, and hung to dry overnight.

The next day, the tresses were visually inspected and felt for stiffness. Tresses treated with the Comparative Samples C and D were flexible with little-to-no stiffness, for example, when their central portions were placed on a support beam, the unsupported ends drooped down. In stark contrast, tresses treated with gels corresponding Batch 1 and Batch 2 felt rigid, and did not bend at all when their central portions were placed on a support beam, indicating excellent hair stiffening and styling performance.

### Example 4

A film made substantially according to the protocol of Example 1 representing Batch 1 was compared to a commercially available styling film product, AVEDA CONTROL TAPE EXTREME STYLE STRIPS hair styling strips (pullulan, modified corn starch, glycerin, camellia oleifera leaf extract, aloe barbadensis leaf extract, linseed extract, hydrolyzed wheat protein, hydrolyzed wheat starch, caprylic/capric triglyceride, fragrance, limonene, linalool, geraniol, eugenol, citronellol, amyl cinnamal, benzyl benzoate, citral, benzyl salicylate, and farnesol).

In-situ formulations were prepared by dissolving 0.3 g of dry film in 12 g of water and stirring until complete dissolution was achieved. Then, 0.4 g of the in situ gel was applied to pre-wetted, pre-combed, eight inch long, ˜4.5 g tresses of European virgin brown hair using a pipette in small portions, evenly from top (swatched end) to bottom (hair tips). The gel was then worked into the hair with fingers going from top to bottom of each tress five times. The tress was then reversed and the same procedure was repeated five more times. At the end, the tress was combed to eliminate knots, smoothed with fingers, and hung to dry overnight.

The treated hair tresses were distributed in pairs to seven expert panelists trained to evaluate performance of cosmetic products on hair. Each panelist evaluated two pairs of tresses, one tress treated with Batch 1 versus one AVEDA hair styling strip control in each pair. The panelists were asked to pick one tress that was more rigid/stiff, combed easier, showed more flaking, felt softer/smoother, combed easier, and had more static flyaways. The evaluation procedures for each of these properties are as follows:
Stiffness: Tresses were gently handled and “felt” for differences in stiffness. Using two fingers, the middles of the swatches were held in a horizontal position to determine which one was bending more than the other. The more rigid one was noted.
Dry comb: The ease of combing was evaluated. The one tress that combed more easily was noted.
Flake: The tress was held at the bound end with one (left) hand, and a fingernail was forcefully run down the length of the tress (right hand). After inspection of both tresses, the one with more flaking was noted.
Feel: The tress that felt silkier/softer/smoother was noted.
Static flyaway's: Each tress was combed at least 5 times and the amounts of flyaway's generated each time were compared. The tress that generated more flyaway's was noted.
   The subjective evaluations were statistically analyzed to identify differences at above 94% confidence level. The findings showed that that Batch 1 was superior to the AVEDA hair styling strip control. For example, Batch 1 significantly outperformed the AVEDA hair styling strips based on stiffness (11/14), dry combability (12/14), feel (13/14), flaking (2/14 (indicating less)), and static flyaway (3/14 (indicating less)). Thus, we unexpectedly found that as little as 24 wt. % (dry film base) of a chitosan derivative added to a styling film was enough to provide excellent styling and conditioning properties, which were in fact superior to a commercial film product.

### Example 5

A film made substantially according to the protocol of Example 1 representing Batch 1 was compared to films made from Comparative Samples A and C.

Batch 1 resulted in a smooth even film (a form preferred by customers) with well balanced mechanical properties, i.e., not too brittle or too flexible. The film did not adhere to itself when folded, which means that it could be packaged as a stack of single-dose films. In contrast, Comparative Sample A resulted in a film with wrinkled, ruffled surface, which pulled easily (too flexible) and adhered to itself when folded similar to a SARAN® wrap film. Due to these properties, films made with Comparative Sample A could not be packaged as a stack of single-dose films without taking extra precautions, such as placing intervening layers between films, which increases packaging costs.

Mechanical properties of dissolvable films are important for in-use properties/handling, as well as for storage and packaging of films. Films should have balanced strength and flexibility so that they are easy to handle and do not pull easily. Thus, the films were subjected to a puncture test to quantify strength and elongation properties.

The test was performed using a TEXTURE ANALYZER TA x-2i instrument. A round-shaped metal probe (½″ in diameter) was used to puncture the film until it was broken. The following conditions were used to conduct the test: measure force in compression test mode, 2.1 mm/sec. test speed, 10 g trigger force. Properties of each 2 mil thick film were measured twice. The film rupture force and film stretching distance (elongation) during the puncture were recorded and summarized in TABLE 3.

TABLE 3

| | Average | Average Film | % Elonga- |
|---|---|---|---|
| | Breaking | Elongation, | tion v. |
| | Force, g | mm | Thickness |
| Batch 1 | 1031 | 1.6 | 80 |
| Comparative Sample A | 3298 | 5.7 | 285 |
| Comparative Sample C | 915 | 1.45 | 72.5 |

The film of the invention, Batch 1, showed optimized mechanical properties and resulted in smooth good looking films with mechanical properties that were close to the comparative METHOCEL hydroxypropyl methylcellulose film, Comparative Sample C, a film which had excellent mechanical properties and aesthetics, but did not provide measurable styling/stiffness (Example 3).

### Example 6

Tactile properties are fundamentally important to consumer preferences. A subjective in-hands study was conducted to compare in-hands properties of films made substantially according to the protocol of Examples 1 and 2 to commercially available OSIS SHOCKFROSTER hair styling strips (modified corn starch, PVP, water, propylene glycol, octylacrylamide/acrylates/butylaminoethyl/methacrylate copolymer, aminomethyl propanol, aluminum starch octenylsuccinate, fragrance, benzyl salicylate, limonene, butylphenyl methylproprional, linalool, and Red 40).

Dissolution rates were measured using the Hand Rubbed Dissolution Test that simulates real-life usage conditions. A 2 cm×3 cm piece of dissolvable film is placed in the palm of the operator's left hand. 2 ml of water are added and the operator rubs the film with the water using two fingers of the right hand in a circular motion (each circle taking approximately one second) until the film is completely dissolved. The dissolution times (average of two measurements) are determined.

Using the Hand Rubbed Dissolution Test, Batch 1 dissolved in 5 (±2) seconds; Batch 2 dissolved in 5 (±2) seconds; Comparative Sample A dissolved in 10 (±2) seconds; Comparative Sample B dissolved in 6 (±2) seconds; Comparative Sample C dissolved in 4 (±2) seconds; Comparative Sample D dissolved in 3.5 (±2) seconds; and the OSIS SHOCKFROSTER hair styling strips dissolved in 15 (±2) seconds.

Thus, films of the present invention performed significantly better than the commercial control (OSIS film). They also showed dissolution times that were close to ones of instantaneously dissolving but non-styling comparative examples (C) and (D). Any improvement in dissolution time is important, as a relatively faster dissolution time relates to positive consumer experience.

### Example 7

Viscosity is yet another important tactile property to consumers. Viscosities were determined for films made substantially according to the protocol of Example 1 (representing Batch 1), and Example 2 (representing Comparative Samples A, B, and C), as well as commercially available OSIS SHOCKFROSTER hair styling strips (see Example 6 for ingredients), AVEDA CONTROL TAPE EXTREME STYLE STRIPS hair styling strips (see Example 4 for ingredients), and SMART H₂O STYLING STRIPS hair styling strips (PVP, modified corn starch, fragrance phenoxyethanol, dimethicone, amodimethicone, methylparaben, C12-14 SEC Pareth 7, C12-14 SEC Pareth 5, ethylparaben, butylparaben, laureth-4, laureth-23, and isobutylparaben).

Viscosities of the in situ gels (prepared by dissolving 0.3 g of dry film in 12 g of water and stirring until complete dissolution) were measured. The Comparative Sample C and all three commercial products gave water-thin in situ gels, which is inconvenient to the consumer and may lead to the loss of some product while still in hands before it gets applied to the hair.

Batch 1, Comparative Sample A, and Comparative Sample B resulted in thicker gels, which are closer to the conventional non-film styling gel products and easier to handle and apply. The viscosities measured using a Brookfield viscometer, model LVDVII+, spindle #60 at 60 rpm and 22° C. are listed in TABLE 4.

TABLE 4

| Film | Viscosity (cps) |
|---|---|
| Batch 1 | 29 |
| Comparative Sample A | 117 |
| Comparative Sample B | 71 |
| Comparative Sample C | 4 |
| OSIS SHOCKFROSTER hair styling strips | 7 |
| (comparative) | |
| AVEDA CONTROL TAPE EXTREME | 4.5 |
| STYLE STRIPS hair styling strips | |
| (comparative) | |
| SMART H₂O STYLING STRIPS hair styling | 8.5 |
| strips (comparative) | |

### Example 8

Tactile properties are fundamentally important to consumer preferences. A subjective in-hands study was conducted to compare in-hands properties of a film made substantially according to the protocol of Example 1 representing Batch 1 to a film made substantially according to the protocol of Example 2 representing Comparative sample A.

Ten panelists participated in this study, with each panelist being asked to compare the ease of dissolution and in-hands tackiness of the respective films and choose one that dissolved faster/easier and felt less tacky. Ten out of ten panelists concluded that the film of the present invention (Batch 1) was easier to dissolve, and nine out of ten believed that Batch 1 felt less tacky than the Comparative Sample A.

All ten panelists noticed that Comparative Sample A yielded thicker gels. Several panelists noted that Comparative Sample A resulted initially in stringy, clumpy gel particles that required additional time to achieve smoother gel texture, which corroborates the findings in Example 6.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A personal care dissolvable film, comprising:
less than about 30 weight percent water-soluble chitosan derivative and greater than about 40 weight percent of a water soluble film forming agent comprising at least one of cellulose derivatives or pullulans.

2. The personal care dissolvable film of claim 1, wherein the water soluble film forming agent is a pullulan.

3. The personal care dissolvable film of claim 1, wherein the water soluble film forming agent is a cellulose ether based polymer.

4. The personal care dissolvable film of claim 1, wherein the water soluble film forming agent is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.

5. The personal care dissolvable film of claim 1, wherein the water soluble film forming agent is hydroxypropyl methylcellulose.

6. The personal care dissolvable film of claim 1, wherein the water soluble film forming agent is present in an amount from about 40% to about 95% by weight of the dry film.

7. The personal care dissolvable film of claim 1, wherein the water-soluble chitosan derivative is a chitosan salt of pyrrolidone carboxylic acid.

8. The personal care dissolvable film of claim 1, wherein the water-soluble chitosan derivative is present in a range from about 5 weight percent to about 30 weight percent.

9. The personal care dissolvable film of claim 1, wherein the water-soluble chitosan derivative is present in a range from about 10 weight percent to about 25 weight percent.

10. The personal care dissolvable film of claim 1, wherein the water-soluble chitosan derivative is present in a range from about 20 weight percent to about 30 weight percent.

11. The personal care dissolvable film of claim 1, wherein the water-soluble chitosan derivative is present in a range from about 22 weight percent to about 27 weight percent.

12. The personal care dissolvable film of claim 1, further comprising a plasticizer comprising at least one of a lipid, a polyol, an acid, a polyester, or organopolysiloxane.

13. The personal care dissolvable film of claim 12, wherein the plasticizer is a polyol plasticizer.

14. The personal care dissolvable film of claim 12, wherein the plasticizer is present in an amount from about 0.01 to about 30 weight percent.

15. The personal care dissolvable film of claim 1, further comprising at least one of cosmetically acceptable additional film forming agents, emollients, moisturizers, conditioners, oils, sunscreens, surfactants, emulsifiers, preservatives, rheology modifiers, colorants, preservatives, pH adjustors, propellants, reducing agents, fragrances, foaming or de-foaming agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides, or an active ingredient selected from skin care actives, nail care actives, or hair care actives.

16. A gel, comprising:
the personal care dissolvable film of claim 1; and
an aqueous component present in an amount sufficient to dissolve the film.

17. The gel of claim 16, wherein the gel has ratio of dry film to water in a range from about 1:10 to about 1:50.

18. The gel of claim 16, wherein the gel has ratio of dry film to water of about 1:40.

19. The gel of claim 16, wherein the gel as a Brookfield viscosity of greater than 10 cps at 25° C.
